(19) United States
(12) Patent Application Publication
(10) Pub. No.: US 2007/0148162 A1
(43) Pub. Date: Jun. 28, 2007

(54) Molecule which binds cd80 and cd86

(76) Inventors: Ranjit Bhardwaj, Berlin (DE); Birgit Vogt, Berlin (DE); Ahmed Sheriff, Berlin (DE)

Correspondence Address: JACOBSON HOLMAN PLLC, 400 SEVENTH STREET N.W., SUITE 600, WASHINGTON, DC, 20004, US

(21) Appl. No.: 10/546,727

(22) PCT Filed: Feb. 26, 2004
(86) PCT No.: PCT/EP04/01916

§ 371 Date: Jul. 24, 2006

(30) Foreign Application Priority Data
Feb. 27, 2003 (EP) 03004446.5

(51) Int. Cl. A61K 39/395; C07K 16/42; C12N 5/06

(52) U.S. Cl. 424/131.1; 530/387.2; 435/327

(57) ABSTRACT

The present invention relates to the identification of molecules, which are specific to CD80 and CD86 antigens. Also preferably, such antibodies are capable of inhibiting the binding of CD28 and CTLA4 to those receptors. Those molecules are able to inhibit T cell mediated immune reactions.

### FIELD OF THE INVENTION

The present invention relates to compounds which are antibodies, antibody fragments, scFv fragment of an antibody, but do not bind specificially to CD40. Also, the present invention relates to pharmaceutical compositions comprising these, e.g. for the treatment of autoimmune disorders, and the prevention of organ rejection or allergies.

### BACKGROUND OF THE INVENTION

The clinical interface between immunology, hematology, and oncology has long been appreciated. Many conditions treated by the hematologist or oncologist have either an autoimmune or immuno-deficient component to their pathophysiology that has led to the widespread adoption of immunosuppressive medications by hematologists, whereas oncologists have sought immunologic adjuvants that might enhance endogenous immunity to tumors. To date, these interventions have generally consisted of nonspecific modes of immunosuppression and immune stimulation. In addition to the limited efficacy of these interventions, toxicities secondary to their nonspecificity have also limited their overall success. Therefore, alternative strategies have been sought.

Elucidation of the functional role of a rapidly increasing number of cell surface molecules has contributed greatly to the integration of immunology with clinical hematology and oncology. Nearly 200 cell surface antigens have been identified on cells of the immune and hematopoietic systems. These antigens represent both lineage-restricted and more widely distributed molecules involved in a variety of processes, including cellular recognition, adhesion, induction and maintenance of proliferation, cytokine secretion, effector function, and even cell death. Recognition of the functional attributes of these molecules has fostered novel attempts to manipulate the immune response. Although molecules involved in cellular adhesion and antigen-specific recognition have previously been evaluated as targets of therapeutic immunologic intervention, recent attention has focused on a subgroup of cell surface molecules termed co-stimulatory molecules.

Co-stimulatory molecules do not initiate but rather enable the generation and amplification of antigen-specific T-cell responses and effector function.

Recently, one specific co-stimulatory pathway termed B7: CD28 has been studied by many research groups because of its significant role in B-and T-cell activation. Since this ligand: receptor pathway was discovered four years ago, a large body of evidence has accumulated suggesting that B7: CD28 interactions represent one of the critical junctures in determining immune reactivity versus anergy.

In particular, the role of the human B7 antigens, i.e., human B7.1 (CD80) and B7.2 (CD86), has been reported to play a co-stimulatory role in T-cell activation.

1. CD80 and CD86 Co-Stimulatory Role in T Cell Activation

The elaboration of a successful immune response depends on a series of specific interactions between a T cell and an antigen presenting cell. Although the essential first step in this process depends upon the binding of antigen to the T cell receptor, in the context of the MHC class II molecule, this interaction alone is not sufficient to induce all the events necessary for a sustained response to a given antigen. The involvement of certain other co-stimulatory molecules is necessary. The homodimers CD28 and CTLA-4 expressed on T cells, together with B7.1 (CD80) and B7.2 (CD86) expressed on antigen presenting cells, are major pairs of costimulatory molecules necessary for a sustained immune response. It can be shown in vitro that the absence of these co-stimulatory signals leads to an aborted T cell activation pathway and the development of unresponsiveness to the specific antigen, or anergy. Achievement of in vivo tolerance constitutes a mechanism for immunosuppression and a viable therapy for organ transplant rejection and for the treatment of autoimmune diseases. This has been achieved in experimental models following the administration of CTLA-4Ig.

The molecules B7.1 (CD80) and B7.2 (CD86) can bind to either CD28 or CTLA-4, although B7.1 (CD80) binds to CD28 with a Kd of 200 nm and to CTLA-4 with a 20-fold higher affinity.

B7.1 is expressed on activated B cells and interferon induced monocytes, but not resting B cells. B7.2 (CD86), on the other hand, is constitutively expressed at very low levels on resting monocytes, dendritic cells and B cells, and its expression is enhanced on activated T cells, NK cells and B lymphocytes. Although B7.1 and B7.2 can be expressed on the same cell type, their expression on B cells occurs with different kinetics. Further analysis at the RNA level has demonstrated that B7.2 mRNA is constitutively expressed, whereas B7.1 mRNA is detected four hours after activation and initial low levels of B7.1 protein are not detectable until 24 hours after stimulation. CTLA-4/CD28 counter receptors, therefore, may be expressed at various times after B Cell activation.

The current data supports the two-signal hypothesis that both a TCR and co-stimulatory signal are necessary for T cell expansion, lymphokine secretion and the full development of effector function. The failure to deliver the second signal results in the inability of T cells to secrete IL-2 and renders the cell unresponsive to antigen.

Structurally, both B7.1 (CD80) and B7.2 (CD86) contain extracellular immunoglobulin superfamily V and C-like domains, a hydrophobic transmembrane region and a cytoplasmic tail. Both B7.1 and B7.2 are heavily glycosylated. B7.1 is a 44-54 kD glycoprotein comprised of a 223 amino acid extracellular domain, a 23 amino acid transmembrane domain, and a 61 amino acid cytoplasmic tail. B7.1 (CD80) contains 3 potential protein kinase phosphorylation sites. B7.2 (CD86) is a 306 amino acid membrane glycoprotein. It consists of a 220 amino acid extracellular region, a 23 amino acid hydrophobic transmembrane domain and a 60 amino acid cytoplasmic tail.

Although both B7.1 (CD80) and B7.2 (CD86) genes are localized in the same chromosomal region, these antigens do not share a high level of homology.

The overall homology between B7.1 and B7.2 is 26% and between murine B7.1 and human B7.1 is 27%. Although alignment of human B7.1 human B7.2 and murine B7.1 sequences shows few stretches of lengthy homology, it is known that all three molecules bind to human CTLA-4 and CD28. Thus, there is most likely a common or closely homologous region shared by the three molecules that may be either contiguous or conformational. This region may constitute the binding site of the CD80 and CD86 molecules to their counter-receptors. Antibodies raised against these epitopes could potentially inhibit the interaction of B7 with its counter-receptor on the T cell. Furthermore, antibodies that cross-reacted with this region on both CD80 and CD86 molecules would potentially have practical advantages over antibodies directed against CD80 or CD86 separately.

2. Blockade of the B7/CD28 Interaction

Blocking of the B7/CD28 (CD80 and/or CD86) interaction offers the possibility of inducing specific immunosuppression, with potential for generating long lasting antigen-specific therapeutic effects. Antibodies or agents that temporarily prevent this interaction may be useful, specific and safe clinical immunosuppressive agents, with potential for generating long term antigen-specific therapeutic effects.

Antibodies to either B7.1 (CD80) or B7.2 (CD86) have been shown to block T cell activation, as measured by the inhibition of IL-2 production in vitro. CTLA-4Ig fusion protein and anti-CD28 Fabs were shown to have similar effects on the down regulation of IL-2 production.

In vivo administration of a soluble CTLA-4Ig fusion protein has been shown to suppress T cell dependent antibody responses in mice and, furthermore, larger doses were also able to suppress responses to a second immunization, demonstrating the feasibility of this approach for the treatment of antibody mediated autoimmune disease. In addition, CTLA-4Ig was able to prevent pancreatic islet cell rejection in mice by directly inhibiting the interaction of T cells and B7.1/B7.2 antigen presenting cells. In this case, long term donor specific tolerance was achieved.

3. Recombinant Phage Display Technology for Antibody Selection

To date, no monoclonal antibodies, which cross-react with both CD80 and CD86 have been reported. No monoclonal antibodies which are specific to CD80 or CD86 and which also recognize specific sites on these molecules which are restricted to co-activation receptor CD28 binding or CTLA-4 receptor binding, have been reported. As discussed above, such antibodies would potentially be highly desirable as immunosuppressants.

Phage display technology is beginning to replace traditional methods for isolating antibodies generated during the immune response, because a much greater percentage of the immune repertoire can be assessed than is possible using traditional methods. This is in part due to PEG fusion and electrofusion inefficiency, chromosomal instability, and the large amount of tissue culture and screening associated with hybridoma production. Phage display technology, by contrast, relies on molecular techniques for potentially capturing the entire repertoire of immunoglobulin genes associated with the response to a given antigen.

This technique was first described by Barbas et al (1) and, independently by Greg Winter's group (2). Essentially, immunoglobulin heavy chain genes are PCR amplified and cloned into a vector containing the gene encoding the minor coat protein of the filamentous phage M13 in such a way that a heavy chain fusion protein is created. The heavy chain fusion protein is incorporated into the M13 phage particle together with the light chain genes as it assembles. Each recombinant phage contains, within its genome, the genes for a different antibody Fab or ScFv molecule, which it displays on its surface. Within these libraries, in excess of 106 different antibodies can be cloned and displayed. The phage library is panned on antigen coated microtiter plate wells or immunotubes, non-specific phage are washed off, and antigen binding phage are eluted. The genome from the antigen-specific clones is isolated and the gene is excised, so that antibody can be expressed in soluble Fab form for further characterization. Once a single Fab is selected as a potential therapeutic candidate, it may easily be converted to a whole antibody. US-A-2002-0150559 discloses a ligand capable of binding to the human CD40 antigen and to the human CD86 antigen and optionally to the CD80 antigen. This antibody, having the combination of the two binding sides, can be used to prevent or treat diseases or conditions in which the activation of T-cells is involved, including transplant rejection, multiple sclerosis, psoriasis, rheumatoid arthritis and systemic lupus erythematosus.

WO-A-01/89567 discloses antibodies which are specific to B7 antigens (CD80 and/or CD86). These antibodies are also capable of inhibiting the binding of B7 to a CD28 receptor. These compounds are used as immunosuppressants, i.e. to block antigen driven immune responses, to treat autoimmune diseases such as psioriasis, rheumatoid arthritis, systemic lupus erythematosus (SLE), type 1 diabetes mellitus, idiopathic thrombocytopenia purpura (ITP), allergy, inflammatory bile disease, and to prevent organ rejection.

Evidence is presented herein for the identification of antibodies which recognize sites on the CD80 and CD86 antigen.

### SUMMARY AND OBJECTS OF THE INVENTION

An object of the invention is to identify novel compounds, preferably an antibody, which specifically binds to human CD80 and CD86 antigen but not to human CD40. More specifically, it is an object of the invention to identify antibodies which are specific to human CD80 and CD86 antigen and which are also capable of inhibiting the binding of CD28 receptors.

It is another object of the invention to identify monoclonal antibodies and chimerized, primatized or humanized forms thereof which recognize specific sites on the human B7.1 antigen and which inhibit T cell proliferation and which function as effective immunosuppressants. It is another object of the invention to provide monoclonal antibodies and chimerized, primatized or humanized forms thereof which inhibit antigen driven responses in donor spleen cell cultures, e.g., antigen specific IgG responses and cell proliferation.

It is another specific object of the invention to identify particular monoclonal antibodies specific to human CD80 and CD86 antigen and chimerized, primatized or humanized forms thereof having advantageous properties, i.e., affinity, immunosuppressive activity, which are useful as therapeutics. More specifically, these antibodies are to be used, e.g., as immunosuppressants, i.e., e., to block antigen driven immune responses, to treat autoimmune diseases such as psoriasis, rheumatoid arthritis, systemic erythematosus (SLE), type 1 diabetes mellitus, idiopathic thrombocytopenia purpura (ITP), allergy, inflammatory bile disease, and to prevent organ rejection.

It is another object of the invention to provide pharmaceutical compositions containing one or more monoclonal antibodies specific to human CD80 and CD86 antigen, and a pharmaceutically acceptable carrier or excipient. These compositions will be used, e.g., as immunosuppressants to treat autoimmune diseases, e.g., idiopathic thrombocytopenia purpura (ITP) and systemic lupus erythematosus (SLE), to block antigen driven immune responses, and to prevent organ rejection in transplant recipients.

It is another object of the invention to provide novel methods of therapy by administration of therapeutically effective amounts of one or more or monoclonal antibody, which specifically binds to human CD80 and CD86 antigen. Such therapeutic methods are useful for treatment of diseases treatable by inhibition of the B7: CD28 pathway, e.g., autoimmune diseases such as idiopathic thrombocytopenia purpura (ITP), systemic lupus erythematosus (SLE), type 1 diabetes mellitus, psoriasis, rheumatoid arthritis, multiple sclerosis, aplastic anemia, as well as for preventing rejection in transplantation subjects.

It is still another object of the invention to provide transfectants, e.g., CHO cells, which express at least the variable heavy and light domains of monoclonal antibodies specific to the human CD80 and CD86 antigen.

The compound of the invention binds both to human CD80 (B7.1) and CD86 (B7.2) but not to human CD40, and which compound inhibits binding of CD28 to human CD80 or CD86, and which does not comprise the extracellular domain of CTLA4 or CD28. The molecule of the invention is an antibody or fragment thereof comprising an antigen-binding site to both, human CD80 and CD86. In particular, the molecule of the invention is a monoclonal antibody comprising an antigen-binding site to both, human CD80 and CD86 but not human CD40. In another embodiment the molecule of the invention is a monoclonal antibody comprising an antigen-binding site to both, human CD80 and CD86 and and is obtainable by immunizing, in particular transgenic, vertebrates, most preferably mice, rats, guinea pigs, hamsters, monkeys, pigs, goats, chicken, cows, horses and rabbits. Preferably, the molecule is a monoclonal antibody containing an antigen-binding site to both, human CD80 and CD86 and which has been produced after immunizing transgenic vertebrates, most preferably mice, rats, guinea pigs, hamsters, monkeys, pigs, goats, chicken, cows, horses and rabbits. In a further embodiment the molecule of the invention is a monoclonal antibody by immunizing humanized vertebrates, preferably those vertebrates mentioned above. In a specific embodiment of the invention, the molecule is a monoclonal antibody comprising an antigen-binding site to both, human CD80 and CD86 and which has been produced by immunizing immunedefective mice (as e.g. SCID or nude mice) repopulated with vital immune cells (e.g. of human origin; as e.g. SCID-hu mice).

The compound of the invention is an antibody, which can be a recombinant antibody (as e.g. single chain antibody—scAb or scFv; bispecific antibody, diabody) which binds to both, human CD80 and CD86, in particular by comprising the antigen-binding site of an antibody which is cross-reactive with CD80 and CD86 but not with human CD40. In particular, the antibody is a humanized antibody.

Subject matter of the invention is also a host cell producing, most preferably stable, the molecule of the invention.

In another embodiment the present invention discloses at least one recombinant vector comprising the nucleotide sequences encoding the molecule or fragments of the molecule according to the invention, operably linked to regulating sequences capable of expressing the antibody of the invention in a host cell, preferably as a secretory protein.

The present invention discloses furthermore tools for carrying out the invention. One tool is e.g. a host transfected with the vector of the invention, preferably a stably transgenic host; a prokaryotic or eukaryotic cell line producing a recombinant antibody according to the invention; a eukaryotic organism, except human beings, such as animals like mammals, a plant or a fungus, producing a recombinant antibody according to the invention.

The invention furthermore discloses a method of producing the compound of the invention in a recombinant method. The compound is capable of binding to both, the human CD80 and CD86 antigen but not CD40, comprising culturing a host cell and isolating the binding molecule from the culture medium and/or the producing cell.

The compound of the invention can be used in a method for inhibiting immunologic responses by treating antigen presenting cells with the CD80/CD86 -binding molecules of the invention.

A pharmaceutical composition for induction of T cell tolerance or inhibition of T cell activation is disclosed, which composition is comprising a therapeutically effective amount of the compound according to the invention and a pharmaceutically acceptable carrier.

The compound of the invention can be used in a method for treating T cell mediated immune responses, a method for preventing allograft transplant rejection, a method for preventing xeno-transplant rejection and/or graft-versions-host-disease, a method for the induction of T cell tolerance or inhibition of T cell activation, a method for the induction of allo-transplant or xeno-transplant tolerance or inhibition of T cell activation, a method for preventing or treatment of autoimmune diseases, a method for preventing or treatment of allergies, a method for treating T cell mediated immune responses to gene therapy vectors, vehicles or expression products, a method for treating T cell mediated immune responses to therapeutic molecules, the method comprising administering to a patient in need of such treatment a therapeutically effective amount of a pharmaceutical composition of the invention.

### DETAILED DESCRIPTION OF THE INVENTION

As described above, the present invention relates to the identification of monoclonal antibodies or chimerized, primatized or humanized forms thereof which are specific to human CD80 and CD86 antigen but not to human CD40 and which are capable of inhibiting the binding of CD28 receptor to CD80 and CD86. Blocking of the primary activation site between CD28 and CD80/CD86 while allowing the combined antagonistic effect on positive co-stimulation with an agnostic effect on negative signalling will be a useful therapeutic approach for intervening in relapsed forms of autoimmune disease. Identified antibodies have demonstrated the ability to block T cell proliferation in excess of 80%. These antibodies possess high affinity to human CD80 and CD86 and may be used as immunosuppressants.

As noted, one method for generating anti-B7 antibodies involves recombinant phage display technology. This technique is generally described above. Essentially, this will comprise synthesis of recombinant immunoglobulin libraries against CD80 and CD86 antigen displayed on the surface of filamentous phage and selection of phage which secrete antibodies having high affinity to CD80 and CD86 antigen. As noted above, preferably antibodies will be selected which bind to both human CD80 and CD86 but not to human CD40.

The second method involves the immunization of vertebrates, most preferably mice, rats, hamsters, guinea pigs, pigs, chicken, cows and horses against human CD80 and CD86 antigen. Such antibodies will be chimerized or humanized to be used as therapeutics in human beings according to known methods of Co MS and Queen C (3). Preferably, mice and even more preferably, humanized vertebrates especially mice, birds or cows are used for this purpose. Humanized animals have the inherent advantage of producing human antibodies against all substances foreign to the corresponding animal.

The significant advantage of antibodies obtained from humanized vertebrates is that these animals recognize also many human proteins as foreign and produce, therefore, many high affinity antibodies to desired human antigens, e.g. human surface proteins and cell receptors. Moreover, because the Ig genes of the humanized animals are phylogenetically identical to human Ig genes, the resultant antibodies exhibit a very high degree of amino acid homology to those produced in humans.

Essentially, human CD80 and CD86 antigens are administered to vertebrates, B cells are isolated therefrom, e.g., lymph node biopsies are taken from the animals, and B lymphocytes are then fused with myeloma cells using polyethylene glycol (PEG) or electrofusion. Hybridomas secreting antibodies which bind human CD80 and CD86 antigen are then identified.

Antibodies which bind to both CD80 and CD86 are desirable because such antibodies potentially may be used to inhibit the interaction with their counter-receptor CD28.

Given that both human CD80 and CD86 bind to human CTLA-4 and CD28, there is at least one common or homologous region to which antibodies were raised.

The disclosed invention involves the use of an animal, which is primed to produce a particular antibody. Animals which are useful for such a process include, but are not limited to, the following: mice, rats, guinea pigs, hamsters, monkeys, pigs, goats, chicken, cows, horses and rabbits.

A preferred means of generating human antibodies using SCID mice is disclosed in U.S. Pat. No. 5,811,524.

Mice were elected to immunize mice against human CD80 and CD86 antigen using recombinant soluble CD80Ig and CD86Ig antigen. However, the particular source of human CD80 and CD86 antigen is not critical, provided that it is of sufficient purity to result in a specific antibody response to the particular administered antigen.

The human CD80 and CD86 antigen genes have been cloned, and sequenced, and therefore may readily be manufactured by recombinant methods.

Preferably, the administered human CD80 and CD86 antigen will be administered in soluble form, e.g., by expression of a CD80 and CD86 gene which has its transmembrane and cytoplasmic domains removed, thereby leaving only the extracellular portion, i.e., the extracellular superfamily V and C-like domains.

The vertebrates will be immunized with the CD80 and CD86 antigen, preferably a soluble form thereof, under conditions which result in the production of antibodies specific thereto. Preferably, the soluble human CD80 and CD86 antigen will be administered in combination with an adjuvant, e.g., Complete Freund's Adjuvant (CFA), Alum, Saponin, or other known adjuvants, as well as combinations thereof. In general, this will require repeated immunization, e.g., by repeated injection, over several months.

For example, administration of soluble CD80 antigen (CD80Ig) to mice was effected in adjuvant, with booster immunizations, over a 3 to 4 month period, with resultant production of serum containing antibodies which bound human CD80 antigen. Afterwards these mice were immunized with soluble CD86 antigen (CD86Ig) using the same scheme.

After immunization B cells are collected, e.g. from the spleen taken from the immunized animals and B lymphocytes fused with myeloma cells using polyethylene glycol.

Hybridomas, which secrete antibodies, which bind human B7 antigen, e.g. CD80 and/or CD86 are then identified. This may be effected by known techniques. For example, this may be determined by ELISA techniques.

Cell lines, which secrete antibodies having the desired specificity to human CD80 and CD86 antigen, are then subcloned to monoclonality.

In the present invention, Hybridoma supernatants were screened containing antibodies for their ability to bind to soluble CD80 antigen coated plates in an ELISA assay. The supernatants of positive clones were tested on soluble CD86 antigen coated plates in an ELISA assay. Hybridomas positive for both, CD80 and CD86 were subjected to further analyses. In addition, the antibodies were screened for their ability to block B cell/T cell interactions as measured by T cell proliferation assays in a mixed leukocyte reaction (MLR).

Also, affinity purified antibodies were tested for their binding to dendritic cells, which express CD80 and CD86.

Cell lines, which express antibodies, which specifically bind to human B7 antigen, B7.1 (CD80) antigen and/or B7.2 (CD86) antigen are then used to clone variable domain sequences for the manufacture of chimerized, primatized or humanized antibodies.

Essentially, this entails extraction of RNA therefrom, conversion to cDNA, and amplification thereof by PCR using Ig specific primers.

The cloned mice variable genes are then inserted into an expression vector, which contains human heavy and light chain constant region genes.

As discussed, lead candidate monoclonal antibodies were selected which specifically bind CD80 and CD86 antigens.

The antibodies can be used either in their native form, or as part of an antibody/chelate (e.g. with PEG) complex. Additionally, whole antibodies or antibody fragments (Fab2, Fab, Fv, ScFv) may be used.

The amount of antibody useful to produce a therapeutic effect can be determined by standard techniques well known to those of ordinary skill in the art.

The antibodies will generally be provided by standard technique within a pharmaceutically acceptable buffer, and may be administered by any desired route.

Because of the efficacy of the presently claimed antibodies and their tolerance by humans it is possible to administer these antibodies repetitively in order to combat various diseases or disease states within a human.

The anti-CD80 and CD86 antibodies (or fragments thereof) of this invention are useful for inducing immunosuppression, i.e., inducing a suppression of a human's immune system.

Also, the use of such antibodies may be used in conjunction with other treatments that may induce or promote apoptosis such as chemotherapeutics, radioimmunotherapy, and antisense therapy, Examples of suitable chemotherapeutics include alkylating agents such as thiotepa and cyclosphosphamide, alkyl sulfonates such as busulfan, Improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamime nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′, 2″-trichlorotriethylamine; urethan; vindesine; dacarbazine; mannomustine; mitobronitol; mitolactol; pipobroman; gacytosine; arabinoside; cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, andtoremifene (Fareston); and antiandrogens such as flutamide, nilutamide, bicalutamide, leuprolide, and goserelin; and pharmaceutically acceptable salts, acids or derivatives of any of the above.

This invention therefore relates to a method of prophylactically or therapeutically inducing immunosuppression in a human or other animal in need thereof by administering an effective, non-toxic amount of such an antibody of this invention to such human or other animal.

The fact that the antibodies of this invention have utility in inducing immunosuppression indicates that they should be useful in the treatment or prevention of resistance to or rejection of transplanted organs or tissues (e.g., kidney, heart, lung, bone marrow, skin, cornea, etc.); the treatment or prevention of autoimmune, inflammatory, proliferative and hyperproliferative diseases, and of cutaneous manifestations of immunologically medicated diseases (e.g., rheumatoid arthritis, lupus erythematosus, systemic lupus erythematosus, Hashimotos thyroiditis, multiple sclerosis, myasthenia gravis, type 1 diabetes, uveitis, nephrotic syndrome, psoriasis, atopical dermatitis, contact dermatitis, IDM and further eczematous dermatitides, seborrheic dermatitis, Lichen planus, Pemplugus, bullous pemphigus, Epidermolysis bullosa, urticaria, angioedemas, vasculitides, erythema, cutaneous eosinophilias, Alopecia areata, etc.); the treatment of reversible obstructive airways disease, intestinal inflammations and allergies (e.g., inflammatory bile disease, Coeliac disease, proctitis, eosinophilia gastroenteritis, mastocytosis, Crohn's disease and ulcerative colitis), food-related allergies (e.g., migraine, rhinitis and eczema), and other types of allergies.

One skilled in the art will be able, by routine experimentation, to determine what an effective, non-toxic amount of antibody will be for the purpose of inducing immunosuppression. Generally, however, an effective dosage will be in the range of about 0.005 to 10 milligrams per kilogram body weight per day.

The route of administration of the antibody (or fragment thereof) of the invention include, by way of example, oral, parenteral, inhalation and topical. The term parenteral as used herein includes intravenous, intraperitoneal, intramuscular, subcutaneous, rectal or vaginal administration. The subcutaneous and intramuscular forms of parenteral administration are generally preferred.

The daily parenteral and oral dosage regimens for employing compounds of the invention to prophylactically or therapeutically induce immunosuppression, or to therapeutically treat carcinogenic tumors will generally be in the range of about 0.005 to 100 mg per kg body weight, but preferably about 0.005 to 10, milligrams per kilogram body weight per day.

The antibodies of the invention may also be administered by inhalation. By “inhalation” is meant intranasal and oral inhalation administration. Appropriate dosage forms for such administration, such as an aerosol formulation or a metered dose inhaler, may be prepared by conventional techniques. The preferred dosage amount of a compound of the invention to be employed is generally within the range of about 10 to 100 milligrams.

The antibodies of the invention may also be administered topically. By topical administration is meant non-systemic administration and includes the application of an antibody (or fragment thereof) compound of the invention externally to the epidermis, to the buccal cavity and instillation of such an antibody into the ear, eye and nose, and where it does not significantly enter the blood stream. By systemic administration is meant oral, intravenous, intraperitoneal and intramuscular administration. The amount of an antibody required for therapeutic or prophylactic effect will, of course, vary with the antibody chosen, the nature and severity of the condition being treated and the animal undergoing treatment, and is ultimately at the discretion of the physician. A suitable topical dose of an antibody of the invention will generally be within the range of about 1 to 100 milligrams per kilogram body weight daily.

Formulations

While it is possible for an antibody or fragment thereof to be administered alone it is preferable to present it as a pharmaceutical formulation. The active ingredient may comprise, for topical administration, from 0.001% to 10% w/w, e.g. from 1% to 2% by weight of the formulation, although it may comprise as much as 10% w/w but preferably not in excess of 5% w/w and more preferably from 0.1% to 1% w/w of the formulation.

The topical formulations of the present invention, comprise an active ingredient together with one or more acceptable carrier(s) therefor and optionally any other therapeutic ingredients(s). The carrier(s) must be “acceptable” in the sense of being compatible with the other ingredients of the formulation and not deleterious to the recipient thereof.

Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container, which is then sealed and sterilised by autoclaving or maintaining at 90-100° C. for half an hour.

Alternatively, the solution may be sterilised by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturiser such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

The subject anti-CD80/CD86 antibodies or fragments thereof may also be administered in combination with other moieties, which modulate the B7: CD28 pathway. Such moieties include, by way of example, cytokines such as IL-7 and IL10, CTLA4-Ig, soluble CTLA-4 and anti-CD28 antibodies and fragments thereof.

Also, the subject antibodies may be administered in combination with other immunosuppressants. Such immunosuppressants include small molecules such as cyclosporin A (CSA) and FK506; monoclonal antibodies such as anti-tumor necrosis factor alpha (anti-TNFα), anti-CD54, anti-CD 11, anti-CD11a, and anti-IL-1; and, other soluble receptors such as rTNFα and rIL-1.

It will be recognized by one of skill in the art that the optimal quantity and spacing of individual dosages of an antibody or fragment thereof of the invention will be determined by the nature and extent of the condition being treated, the form, route and site of administration, and the particular animal being treated, and that such optimums can be determined by conventional techniques. It will also be appreciated by one of skill in the art that the optimal course of treatment, i.e., the number of doses of an antibody or fragment thereof of the invention given per day for a defined number of days, can be ascertained by those skilled in the art using conventional course of treatment determination tests.

Without further elaboration, it is believed that one skilled in the art can, using the preceding description, utilise the present invention to its fullest extent. The following formulations are, therefore, to be construed as merely illustrative embodiments and not a limitation of the scope of the present invention in any way.

Capsule Composition

A pharmaceutical composition of this invention in the form of a capsule is prepared by filling a standard two-piece hard gelatin capsule with 50 mg of an antibody or fragment thereof of the invention, in powdered form, 100 mg. of lactose, 32 mg. of talc and 8 mg. of magnesium stearate.

Injectable Parenteral Composition

A pharmaceutical composition of this invention in a form suitable for administration by injection is prepared by stirring 1.5% by weight of an antibody or fragment thereof of the invention in 10% by volume propylene glycol and water. The solution is sterilised by filtration.

Eye Drop Composition

Antibody or fragment thereof of the invention 0.5 g.

Methyl Hydroxybenzoate 0.01 g.

Propyl Hydroxybenzoate 0.04 g.

Purified Water to 1 ml.

The methyl and propyl hydroxybenzoates are dissolved in 70 ml purified water at 75° C. and the resulting solution is allowed to cool. The antibody or fragment thereof of the invention is then added, and the solution is sterilised by filtration through a membrane filter (0.022 um pore size), and packed aseptically into suitable sterile containers.

Composition for Administration by Inhalation (1)

For an aerosol container with a capacity of 15-20 ml: mix 10 mg. of an antibody or fragment thereof of the invention with 0.2-0.5% of a lubricating agent, such as polysorbate 85 or oleic acid, and disperse such mixture in a propellant, such as freon, preferably in a combination of (1,2 dichlorotetrafluoroethane) and difluorochloro-methane and put into an appropriate aerosol container adapted for either intranasal or oral inhalation administration.

Composition for Administration by Inhalation (2)

For an aerosol container with a capacity of 15-20 ml: dissolve 10 mg. of an antibody or fragment thereof of the invention in ethanol (6-8 ml.), add 0.1-0.2% of a lubricating agent, such as polysorbate 85 or oleic acid; and disperse such in a propellant, such as freon, preferably in combination of (1.2 dichlorotetrafluoroethane) and difluorochloromethane, and put into an appropriate aerosol container adapted for either intranasal or oral inhalation administration.

The antibodies and pharmaceutical compositions of the invention are particularly useful for parenteral administration, i.e., subcutaneously, intramuscularly or intravenously. The compositions for parenteral administration will commonly comprise a solution of an antibody or fragment thereof of the invention or a cocktail thereof dissolved in an acceptable carrier, preferably an aqueous carrier. A variety of aqueous carriers may be employed, e.g., water, buffered water, 0.4% saline, 0.3% glycine, and the like. These solutions are sterile and generally free of particulate matter. These solutions may be sterilised by conventional, well-known sterilisation techniques. The compositions may contain pharmaceutically acceptable auxiliary substances as required to approximate physiological conditions such as pH adjusting and buffering agents, etc. The concentration of the antibody or fragment thereof of the invention in such pharmaceutical formulation can vary widely, i.e., from less than about 0.5%, usually at or at least about 1% to as much as 15 or 20% by weight, and will be selected primarily based on fluid volumes, viscosities, etc., according to the particular mode of administration selected.

Thus, a pharmaceutical composition of the invention for intramuscular injection could be prepared to contain 1 mI sterile buffered water, and 50 mg. of an antibody or fragment thereof of the invention. Similarly, a pharmaceutical composition of the invention for intravenous infusion could be made up to contain 250 ml of sterile Ringer's solution, and 150 mg of an antibody or fragment thereof of the invention. Actual methods for preparing parenterally administrable compositions are well known or will be apparent to those skilled in the art, and are described in more detail in, for example, Remingto's Pharmaceutical Science, 15th ed., Mack Publishing Company, Easton, Pa., hereby incorporated by reference herein.

The antibodies (or fragments thereof) of the invention can be lyophilized for storage and reconstituted in a suitable carrier prior to use. This technique has been shown to be effective with conventional immune globulins and art-known lyophilization and reconstitution techniques can be employed.

Depending on the intended result, the pharmaceutical composition of the invention can be administered for prophylactic and/or therapeutic treatments. In therapeutic application, compositions are administered to a patient already suffering from a disease, in an amount sufficient to cure or at least partially arrest the disease and its complications. In prophylactic applications, compositions containing the present antibodies or a cocktail thereof are administered to a patient not already in a disease state to enhance the patient's resistance.

Single or multiple administrations of the pharmaceutical compositions can be carried out with dose levels and pattern being selected by the treating physician. In any event, the pharmaceutical composition of the invention should provide a quantity of the altered antibodies (or fragments thereof) of the invention sufficient to effectively treat the patient.

It should also be noted that the antibodies of this invention may be used for the design and synthesis of either peptide or non-peptide compounds. (mimetics) which would be useful in the same therapy as the antibody (4).

### EXAMPLES

1. Human In Vitro Model

Abrogation of unwanted immune reactions can be shown in vitro by reduction of T cell proliferation in mixed leukocyte reactions. This is an in vitro model considered as relevant by persons known in the art.

Dendritic cells from one donor are mixed with T cells from another allogenic donor. Due to different HLA antigens an immune reaction is started and T cells proliferate. The extent of proliferation is an indicator for the severity of the reaction. The same mechanism is true for specific antigens (e.g. autoantigens) in syngenic models.

Allogen Proliferation Assays with Human Dendritic Cells

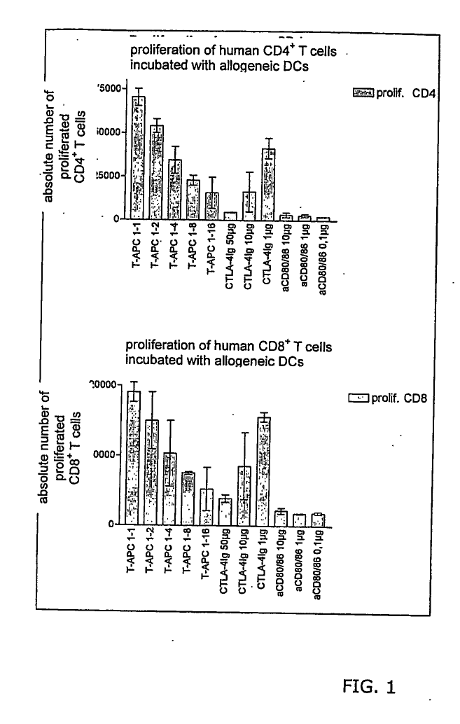

Reduction of proliferation was shown in the human system. The anti CD80 and CD86 antibodies and the CD80/CD86 blocking human CTLA-4Ig were added directly into a mixed leukocyte reaction containing human DCs and allogeneic T cells. Six days later T cell proliferation was analysed. Results are shown in FIG. 1.

2. Mouse In Vivo Model

An accepted in vivo model is the Ovalbumin (OVA) system. In this system, a mouse is sensitised by immunisation with the model antigen OVA. Specific antibody titers can be determined using the ELISA technique. The antibody titers are indicators for the resulting immune reaction. Especially the titers after recontact (boost) with the antigen are important. A decrease in antibody titers is a sign for tolerance induction. Costimulation blocked antigen presenting cells (APC), as e.g. DC or B cells, can be used as vehicles for tolerance induction.

Reduction of Serum Levels of Specific Antibodies by Injection of Costimulation Blocked DC

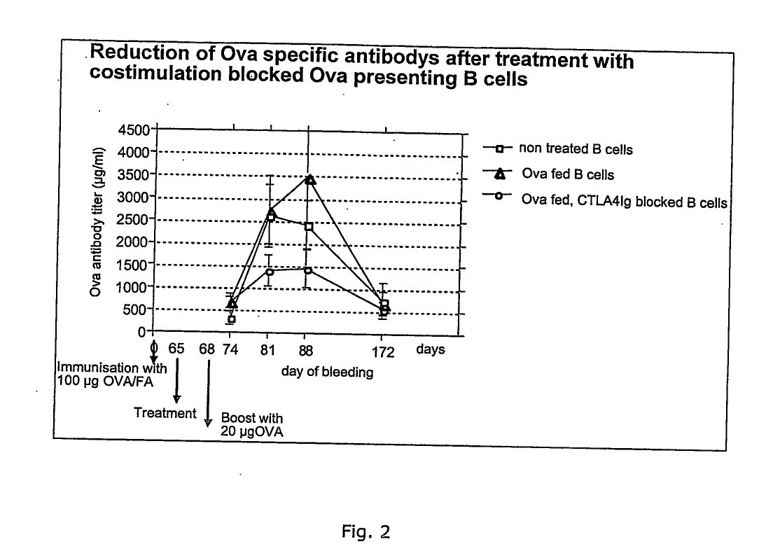

Mice were immunised with ovalbumin and antibody titers (against ovalbumin) were quantified by ELISA. Costimulation blockade was performed by adding CTLA-4Ig protein to the APC prior to injection into mice. Loading the APCs with ovalbumin (OVA) was also done by adding the protein to the APC. Mice were immunised with ovalbumin, treated with cells 2 month later and rechallenged with antigen 3 days afterwards. In this experiment B cells were used as APC. FIG. 2 shows the results.

FIG. 1: Human monocyte-derived DCs from one donor were incubated with MACS sorted CFSE stained T cells from another donor for 6 days. The ratio of T cells to DCs (APCs) was varied from 1:1 up to 1:16. Additionally, the 1:1 ratio setting was supplemented either with different concentrations of human CTLA-4Ig fusion protein (1-50 μg/ml) or blocking anti-CD80 and anti-CD86 antibodies (0.1-10 μg/ml). Proliferation was measured by FACS analysis of CFSE decrease in CD4⁺ or CD8⁺ cells. Absolute cell numbers were calculated using Truecount beads.

This experiment showed the dose-dependent reduction of T cell proliferation through the action of CD80/CD86 blocking biologics. CTLA4Ig blocks both CD80 and CD86 at the same time and leads to abrogation of the immune reaction. Blocking with a combination of anti-CD80 and anti-CD86 antibodies was even more effective. This is due to the higher affinity of those monoclonal antibodies if compared with CTLA-4Ig. Therefore, the antibody blocking both CD80 and CD86 of the present invention will be even more effective than the mixture used in this experiment.

FIG. 2: Specific antibody titers in mice after treatment with B cells and rechallenge. B cells were either untreated, fed with OVA or fed with OVA and CTLA-4Ig (costimulation block). Mice were injected i.v. with 10⁶ cells; animals receiving blocked B cells were treated on two consecutive days. Each group contained 5 mice. Shown is the average antibody titer in μg/ml.

Despite the high rechallenge dose of 20 μg OVA per mouse, animals treated with antigen-specific, costimulation-blocked cells showed a markedly reduced antibody titer. Treatment with costimulation unreduced antigen specific B cells or untreated B cells showed an increased antibody titer.

Based on the results of the experiment in FIG. 1, one can propose that the use of the antibody of the present invention will further reduce the OVA antibody titer depicted in FIG. 2. This is due to the enhanced binding properties of the antibodies and leads to a total inhibition of antibody production even after further recontact.

### REFERENCES

1. Barbas, C. F., 3rd, A. S. Kang, R. A. Lerner, S. J. Benkovic. 1991. Assembly of combinatorial antibody libraries on phage surfaces: the gene III site. Proc Natl Acad Sci USA. 88: 7978-82.
2. Clackson, T., H. R. Hoogenboom, A. D. Griffiths, G. Winter. 1991. Making antibody fragments using phage display libraries. Nature. 352: 624-8.
3. Co, M. S., C. Queen. 1991. Humanized antibodies for therapy. Nature. 351: 501-2.
4. Saragovi, H. U., D. Fitzpatrick, A. Raktabutr, H. Nakanishi, M. Kahn, M. I. Greene. 1991. Design and synthesis of a mimetic from an antibody complementarity-determining region. Science. 253: 792-5.

1. A compound which is an antibody, antibody fragment, Fv fragment, Fab fragment or scFv-fragment of an antibody which binds to both human CD80 (B7.1) and human CD86 (B7.2) but not to human CD40, which compound inhibits binding of CD28 to human CD80 or human CD86, and which compound does not comprise the extracellular domain of CTLA4 or CD28.

2. A host cell producing the compound according to claim 1.

3. A method of producing the compound according to claim 1 binding to both, the human CD80 and CD86 antigen, comprising culturing a host cell and isolating the binding molecule from the culture medium and/or the producing cell.

4. A method for inhibiting immunologic responses by treating isolated antigen presenting cells or cells in culture with the compound according to claim 1.

5. A pharmaceutical composition for induction of T cell tolerance or inhibition of T cell activation, comprising a therapeutically effective amount of the compound according to claim 1 and a pharmaceutically acceptable carrier.

6. Use of the compound according of claim 1 for the manufacturing of a medicament for treating T cell mediated immune responses.

7. Use according to claim 6 for preventing allograft transplant rejection, xeno transplant rejection and graft versus host disease.

8. Use according to claim 6 for the induction of T cell tolerance or inhibition of T cell activation, induction of allo-transplant or xeno transplant tolerance or inhibition of T cell activation.

9. Use according to claim 6 for preventing or treatment of autoimmune diseases, or allergies.

10. Use according to claim 6 for treating T cell mediated immune responses to gene therapy vectors, vehicles or expression products or for treating T cell mediated immune responses to therapeutic molecules.
